(19) United States
(12) Patent Application Publication
(10) Pub. No.: US 2024/0293280 A1
(43) Pub. Date: Sep. 5, 2024

(54) AN ORTHOSIS DEVICE

(71) Applicant: UNIVERSIDAD SANTIAGO DE CALI, Cali (CO)

(72) Inventors: Leonardo Antonio Bermeo Varon, Cali (CO); John Alexander Morales Rodriguez, Cali (CO); William Alberto Rodriguez Criales, Cali (CO); Diana Maritza Quiguanas Lopez, Cali (CO); Edgar Francisco Arcos Hurtado, Cali (CO); John Jairo Villarejo Mayor, Cali (CO)

(73) Assignee: UNIVERSIDAD SANTIAGO DE CALI, Cali (CO)

(21) Appl. No.: 18/573,217

(22) PCT Filed: Jun. 17, 2022
(86) PCT No.: PCT/IB2022/055622

§ 371 Date: Dec. 21, 2023

Related U.S. Application Data
(60) Provisional application No. 63/345,946, filed on May 26, 2022.

(30) Foreign Application Priority Data
Jun. 29, 2021 (CO) NC2021/0008539

(51) Int. Cl. A61H 1/02

(52) U.S. Cl. CPC A61H 1/0288; A61H 2201/1676; A61H 2230/605

(57) ABSTRACT

The present disclosure relates to an orthosis device for providing motor movement to a body part. The device comprises a plurality of sensors to sense a first set of motion signals, a control unit, at least one actuator, and a support structure. The support structure comprises a guide structure comprising a plurality of wires. The control unit generates a second set of motion signals and provides the same to the at least one actuator. The at least one actuator generates a force for movement of the plurality of wires of the guide structure of the support structure. The movement of the plurality of wires help in motor movement of the body part.

### FIELD

The present disclosure generally relates to orthosis. More particularly, the present disclosure relates to an orthosis device for providing motor movement to a body part.

### BACKGROUND

Spinal cord injuries and neurological diseases such as muscular dystrophy, cerebral palsy, and stroke can lead to partial or complete loss of hand mobility. According to the WHO, about 500,000 people each year worldwide suffer from spinal cord injury (SCI) and approximately 15 million people have a stroke. Individuals with this type of injury lose the ability to complete everyday activities like grasping objects, drinking a glass of water, or dressing, which affect their autonomy and emotional state. Rehabilitation and assistive technologies have been proposed to restore hand functionality such as active orthoses, exoskeletons, and robotic gloves. These devices allow assisting to complete hand movements through actuated systems that are directly controlled by the user. The control can be based on On/off control or pattern recognition techniques. In a control system, a trigger signal (defined by a threshold) acts as a switch to control the execution of a specific task. In pattern recognition, movements are associated with patterns represented by features of the signal.

These patterns are previously trained and classified to generate control commands. The most common signals used for orthoses control are electroencephalographic (EEG) and voice signals, electrooculogram (EOG), control switches, and surface electromyography (sEMG). Most orthoses (42.00%) are controlled by the sEMG signal, while 8% are controlled by voice signal and 8.00% by EEG. This is because sEMG based orthosis control provides a natural mapping of the intention for spontaneous muscle movement. Besides, it allows other activities to be performed simultaneously without requiring additional concentration. In individuals with spinal cord injuries, sEMG signals from muscles with remaining voluntary contractions have been used for hand orthosis control. Myoelectric pattern recognition has been widely used to control orthoses and prostheses. Different factors detennine the type of signal processing, such as the electrodes locations, the influence of intrinsic and extrinsic noise-generating artifacts, and the number of electrodes. High-density electrode configurations provide high recognition efficiency. But, it has higher requirements for hardware, processing, and computational cost. Also, this configuration increases the difficulty of dressing and the probability of electrode failure, which makes it mainly used in laboratory environments and rarely in daily use. On the other hand, low density systems are normally used in commercial EMG devices, which have low cost, easy to dress, and acceptable performance. Further, feature sets in the time, frequency, and time-frequency domains have been used to represent myoelectric patterns. Moreover, different learning machine methods such as k-nearest neighbor (KNN), artificial neural networks (ANN), linear discriminant analysis (LDA), support vector machine (SVM). have been used as classification methods. In particular, studies showed that SVM combined with genetic algorithms for optimizing feature selection performed slightly better than LDA and SVM for hand movements classification. However, KNN is computationally efficient and easy to be implemented in hardware, being a suitable option for real-time applications. Nevertheless, most of these works have been evaluated in people without injuries, which shows the need to validate these methods with individuals with SCI. Besides, validation of systems in real-time is required to evaluate their true applicability in daily use. Studies based on real-time sEMG showed better classification by increasing the number of channels. However, in individuals with spinal cord injury, it is recommended to use low-density sEMG, due to muscle atrophy and limitation of the active muscles that induce movement compensations.

Currently, there are a number of solutions for providing an active hand orthosis to improve hand function or for rehabilitation. Some of these solutions attempt to reproduce the hand function, but without the intention of movement, i.e., with pre-programmed routines in the electronic system. Other solutions are not ergonomic or comfortable due to their general design. In most cases, these solutions do not meet the needs of people who need to improve their hand functions because they do not perform the task well, are not specific, are not comfortable, and are not even functional.

### SUMMARY

It is an object of the present disclosure to ameliorate limitation of the prior art by providing an orthosis device which is used for providing motor movement to a body part.

An object of the present disclosure is to provide an orthosis device which allows controlled movement of a body part of a patient.

Another object of the present disclosure is to provide an orthosis device which enables movement of a body part of a patient without time delay.

Yet another object of the present disclosure is to provide an orthosis device which is light weight.

Yet another object of the present disclosure is to provide an orthosis device which is comfortable to use.

Yet another object of the present disclosure is to provide an orthosis device which ha low manufacturing cost.

The present disclosure discloses an orthosis device for providing motor movement to a body part. The device comprises a plurality of sensors, a control unit in communication with the plurality of sensors, at least one actuator in communication with the control unit, and a support structure in communication with the at least one actuator.

The plurality of sensors are configured to be placed on a preselected body part and are further configured to detect a first set of motion signals from the preselected body part. The control unit is configured to receive the first set of motion signals from the plurality of sensors and is further configured to generate a second set of motion signals based on the received first set of motion signals. The at least one actuator is configured to receive the second set of motion signals to be activated. The support structure is configured to provide support to the body part and is further configured to displace the body part in accordance with the at least one actuator, thereby providing motor movement to the body part.

The support structure comprises a guide structure having a plurality of wires to facilitate movement of the body part.

The at least one actuator is configured to provide movement to the support structure in a plurality of states and is further configured to generate a force to displace the guide structure of the support structure, thereby providing movement to the body part. The at least one actuator is a servo motor.

The control unit is configured to receive the first set of motion signals, classify the first set of motion signal into a class of a plurality of classes, and generate a corresponding second set of motion signals based on the class. The plurality of classes corresponds to a plurality of states of movement of the support structure. The plurality of states of movement comprises a state of flexion, a state of expansion, and a relax state.

### BRIEF DESCRIPTION OF DRAWING

FIG. 1 illustrates an exemplary overview of an orthosis device in accordance with the present disclosure;

FIG. 2 illustrates an exemplary orthosis device used for movement of a hand;

FIG. 3 illustrates an exemplary confusion matrix for able-bodied person derived using a control unit of FIG. 1;

FIG. 4 illustrates an exemplary confusion matrix for a patient using the device of the present disclosure;

FIG. 5 illustrates an exemplary support structure in accordance with one embodiment of the present disclosure;

FIG. 6 illustrates an exemplary first portion of a support structure of an orthosis device of the present disclosure;

FIG. 7 illustrates an exemplary second portion of a support structure of an orthosis device of the present disclosure;

FIG. 8 illustrates an exemplary third portion of a support structure of an orthosis device of the present disclosure;

FIG. 9 illustrates an exemplary fourth portion of a support structure of an orthosis device of the present disclosure;

FIG. 10 illustrates the complete structure of the orthosis on a human hand; and

FIG. 11 illustrates exemplary signals indicating a first set of motion signals and a corresponding second set of motion signals detected and generated by the orthosis device in accordance with the present disclosure.

### LIST OF REFERENCE NUMERALS

10—orthosis device
1—support structure
2—actuator
3—control unit
4,5—plurality of sensors
20—plurality of wires
100—first portion of the support structure
21—first protrusion
22—second protrusion
23—first set of holes
24—second set of holes
200—second portion of the support structure
31, 32—plurality of articulation holes
33, 34, 35—second set of holes of the second portion
300—third portion of the support structure
41, 42—plurality of holes of the third portion
43—bottom edge
44—top edge
400—fourth portion of the support structure
51—plurality of guide holes
52—plurality of provisions

### DETAILED DESCRIPTION

Embodiments, of the present disclosure, will now be described with reference to the accompanying drawing.

Spinal cord injuries and neurological diseases such as muscular dystrophy, cerebral palsy, and stroke can lead to partial or complete loss of mobility, especially of hands. To help such people, currently there are a number of solutions for providing an active hand orthosis to improve hand function or for rehabilitation. Some of these solutions attempt to reproduce the hand function, but without the intention of movement, i.e., with pre-programmed routines in the electronic system. Other solutions are not ergonomic or comfortable due to their general design. In most cases, these solutions do not meet the needs of people who need to improve their hand functions because they do not perform the task well, are not specific, are not comfortable, and are not even functional. To overcome the limitation of the existing available device, the present disclosure provides an orthosis device which provides mobility to different body parts, such as hands and legs.

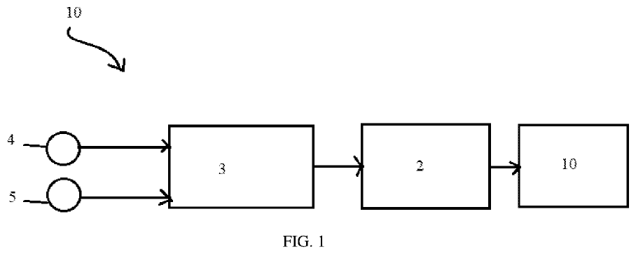

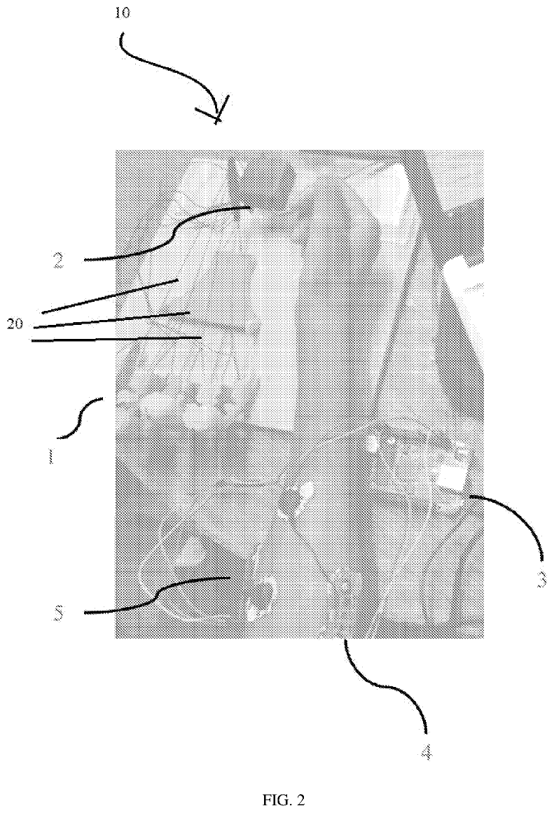

According to a first aspect of the present disclosure, an orthosis device 10 for providing motor movement to a body part is disclosed. FIG. 1 illustrates an exemplary overview of such orthosis device 10 in accordance with the present disclosure. The orthosis device 10 comprises a plurality of sensors 4, 5. The plurality of sensors 4, 5 are configured to be placed on a preselected portion of a body part for which movement is desired. The plurality of sensors 4, 5 are non-invasive sensors 4, 5 and are configured to detect a first set of motion signals. In a preferred embodiment, the plurality of sensors 4, 5 are surface electromyography muscle sensors 4, 5 and are configured to detect a first set of EMG signals from the preselected portion of the body part. For example, if a patient is disabled due to a C5 and C6 spinal trauma that generates muscle atrophy at a distal level of the hand, then to provide motor movement to the hand, the plurality of sensors 4, 5 can be placed at the flexor digitorum superficialis and extensor digitorum muscles of the hand to measure the EMG signals. The EMG signals measured at the flexor digitorum superficialis and extensor digitorum muscles are used for motor movement of the distal part of the hand, such as fingers.

The device 10 further comprises a control unit 3 coupled with the plurality of sensors 4, 5. The control unit 3 is configured to receive the first set of motion signals and further configured to generate a second set of motion signals based on the received first set of motion signals. The control unit 3 comprises at least one processor and a memory attached to the at least one processor. The control unit 3 is configured to process the received first set of motion signals in real-time, namely online process, as well as offline.

In particular, the control unit 3 is configured to classify the first set of motion signals into one of the plurality of classes to generate the second set of motion signals. The plurality of classes corresponds to a plurality of states of movement. Some non-limiting examples of the plurality of states are a state of flexion, a state of expansion, and a relax state. The control unit 3 uses one or more artificial intelligence techniques to generate the second set of motion signals. Accordingly, the control unit 3 is configured to generate a separate second set of motion signals corresponding to the state of flexion, the state of expansion, and the relax state.

In some exemplary embodiments, the processing of the first set of motion signals is performed using Artificial intelligence and Machine learning techniques. While using such techniques, training dataset is generated which is used to train a machine learning model for accurate classification of new data. In an embodiment, the training dataset is generated by measuring the first set of motion signals and the second set of motion signals from an able-bodied person. In such embodiment, a first set of motion signals corresponding to each state of motor movement is measured using the plurality of sensors 4, 5, and a corresponding second set generated by the able-bodied person is measured using another set of sensors. These first set of motion signals and the second set of motion signals along with the corresponding state of the motor movement is stored in the training dataset. The term ‘able-bodied person’ refers to a person who is completely independent in performing different motor movement of all the body parts.

In an exemplary embodiment, the step of processing the first set of motion signals include one or more preprocessing steps and one or more analysis steps. The one or more preprocessing steps comprise a step of eliminating a portion of the first set of motion signals which indicate transition between the one or more motor movements. The one or more analysis steps includes a step to generate overlapping signal for reducing input delay in the first set of motion signals. Hence, the one or more steps of preprocessing and the analysis help in generating analyzed signals which are noise free and delay-free, and are ready for further processing. After the analysis, the analyzed signals are classified using one or more classification techniques. Some non-limiting examples of such classification techniques are ON-OFF control classification and k-nearest neighbors (kNN) technique.

In the ON-OFF control classification, two threshold values are provided for a motion signal received from each of the plurality of sensors 4, 5. Each of the analyzed signals derived from the first set of motion signals is compared with the corresponding two threshold values to classify the analysed signals into one of the plurality of states.

In the kNN technique, a distance between the analysed signals and corresponding training signals from training dataset is compared. For such comparison, four distances are considered namely Euclidean distance, Minkowski distance, Manhattan distance, and Chebyshev distance. A distance with highest efficiency is considered ideal for classification and for generating second set of signals.

In performing the kNN classification, a five-fold stratified shuffle split is used, in which four parts of training dataset work as training data and the fifth part work as validation data in training kNN based machine learning model.

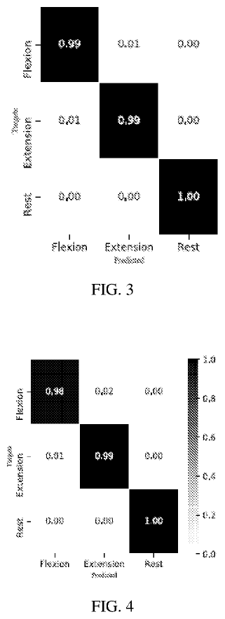

FIG. 3 and FIG. 4 illustrate exemplary confusion matrices for able-bodied person and a patient using the control unit 3 of the device 10 in accordance with the present disclosure, respectively. For the patient, it can be noted a confusion between flexion and extension states, with 2.00% as the highest false positives. For the able-bodied person, the flexion and extension classes showed errors of 1.00%, with false positives between the two classes respectively. The resting class showed 100.00% recognition for the subjects. Hence, the device 10 of the present disclosure is configured to detect and recognize real-time movement of one or more first set of motion signals and further configured to generate a corresponding second set of motion signals for motor movement of the corresponding body part.

The device 10 further comprises at least one actuator 2 and a support structure 1. The at least one actuator 2 is coupled with the control unit 3 and is configured to receive the second set of motion signals generated by the control unit 3. On receiving the second set of motion signals, the at least one actuator 2 is configured to generate a force. The force is used for providing motor movement to the body part. The force generated corresponds to the state of movement of the body part. In an embodiment, the at least one actuator 2 is a servo motor.

The support structure 1 is configured to provide support to the body part for which the movement is desired. In an embodiment, the support structure 1 is configured to provides support to distal part of hand, specifically to fingers of the hand. The motor movement in such embodiment is flexion and relaxation of the fingers, which results in a grabbing action and a releasing action. Accordingly, the at least one actuator 2 is configured to generate force in the clockwise direction and the anticlockwise direction. Hence, the device 10 of the present disclosure is configured to provide the motor movement for grabbing and releasing an object for a patient. This gives control of the movement of the hand to the patient in real-time or near real-time. Hence, the patient, after facing the major injury, feels confident and independent, resulting in improvement of condition of the patient.

In such embodiment, the plurality of sensors 4, 5 are placed at the flexor digitorum superficialis and extensor digitorum muscles of the hand to receive first set of motion signals. For example, the EMG signal for grabbing a cup is generated by the brain of the patient. These are first set of motions signals. The control unit 3 receives these first set of motion signals and generates a second set of motion signals corresponding to the state of flexion. The second set of motion signals are received by the at least one actuator 2. The at least one actuator 2, based on the second set of motion signals, generates a force to move the support structure 1. The support structure 1 is configured to be displaced according to the force generated by the at least one actuator 2. Hence, the support structure 1, which is worn at palm and fingers of the hand of the patient, is configured to move the fingers such that the fingers transform to the flexion, which results in flexion movement of the fingers. In other words, the hand performs grabbing action and the patient grabs a cup.

The device 10 of the present disclosure is used with a patient for a certain period of time, for example for 6 months after an accident. The device 10 helps the patient to regain the control of the movement, which was lost due to the accident.

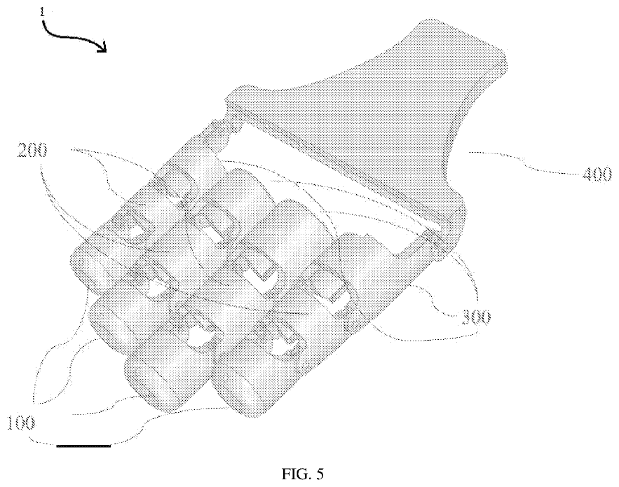

FIG. 5 illustrates an exemplary support structure 1 of the device 10 in accordance with the present disclosure. The exemplary support structure 1 corresponds to a distal part of the hand, specifically to the palm and fingers, of the patient. Such support structure 1 comprises a first portion 100, a second portion 200, a third portion 300, and a fourth portion 400. As shown in FIG. 5, the first portion 100, the second portion 200, the third portion 300, and the fourth portion 400 are attached with each other in a way to provide support to the palm and the fingers of the hand of the patient.

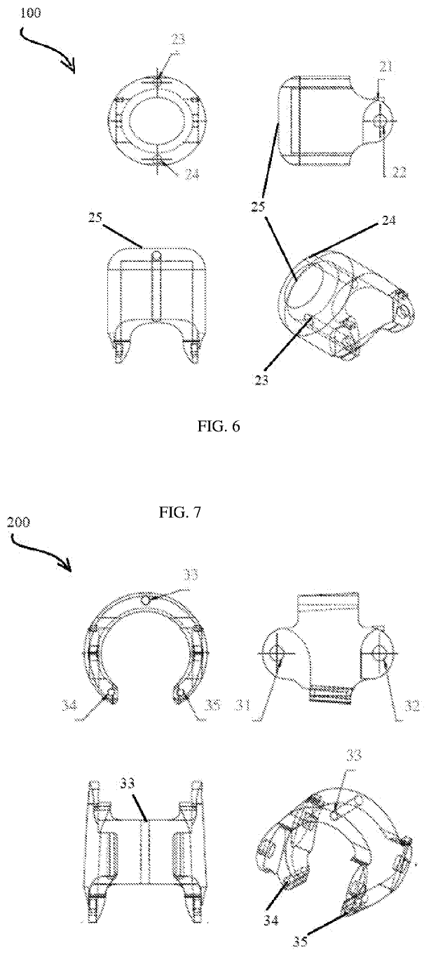

FIG. 6 illustrates an exemplary first portion 100 of the support structure 1 in accordance with one aspect of the present disclosure. The first portion 100 corresponds to tips of the fingers of the hand of the patient. The first portion 100 comprises a top portion to provide support to a distal end of the body part, i.e., finger tips, and to prevent hypertension while movement, a first protrusion 21 and a second protrusion 22 configured to be connected with the second portion 200 and further configured to form a joint, a first set of holes 23 to provide expansion to the body part, and a second set of holes 24 to provide flexion to the body part. In a preferred embodiment, the first set of holes 23 are placed such that they are positioned at back of the corresponding finger and the second set of holes 24 are placed such that they are positioned at front of the corresponding finger.

FIG. 7 illustrates an exemplary second portion 200 of the support structure 1 in accordance with the first aspect of the present disclosure. The second portion 200 of the support structure 1 comprises a plurality of articulation holes 31, 32 configured to connect the first protrusion 21 and the second protrusion 22 of the first portion 100. Hence, the plurality of articulation holes 31, 32 provides stable and flexible connection between the first portion 100 and the second portion 200 of the support structure 1. The second portion 200 further comprises a first set of protrusions to connect to the third portion 300, and a second set of holes 33, 34, 35 configured to provide proper movement to the fingers of the hand. In an embodiment, one of the second set of holes 33 corresponds to the first set of holes 23 of the first portion 100 and help in flexion movement of the finger. The remaining holes of the second set of holes 34,35 correspond to the second set of holes 24 of the first portion 100 and help in the expansion movement of the finger.

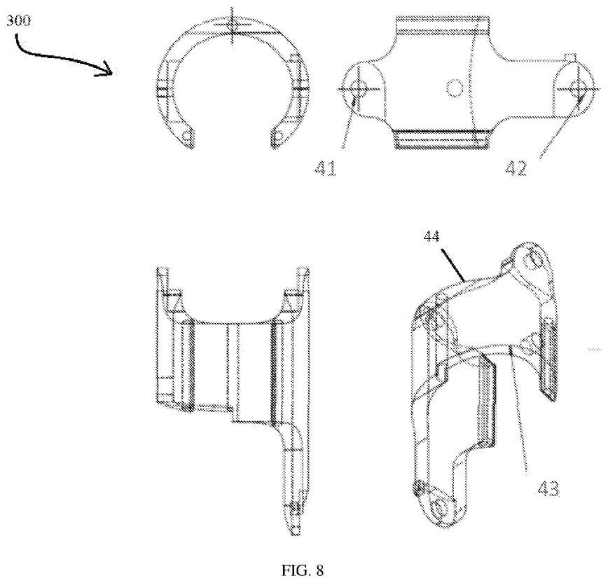

FIG. 8 illustrates an exemplary third portion 300 of the support structure 1 in accordance with the first aspect of the present disclosure. The third portion 300 comprises a top edge 44 having a plurality of protrusions 41, 42 configured to be connected to the first set of holes of the second portion 200, and a bottom edge 43. Hence, the plurality of protrusions 41, 42 provides stable and flexible connection between the second portion and the third portion of the support structure 1. In some embodiments, the bottom edge 43 of the third portion 300 of the index finger and the little finger comprises an extended portion for providing connection with the fourth portion of the support structure 1. The extended portion comprises a protrusion for attachment with the fourth portion. The fourth portion further comprises hollow pipe portions and a back hole corresponding to the second set of holes 34-35, and 33, respectively.

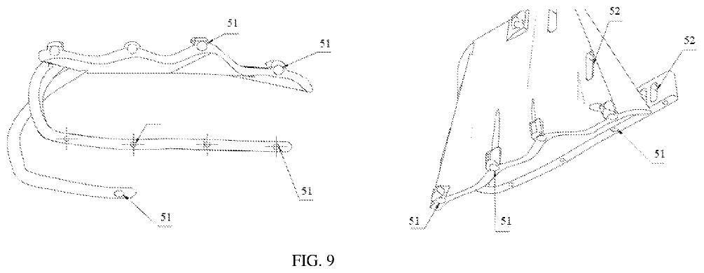

FIG. 9 illustrates an exemplary fourth portion 400 of a support structure 1 of the orthosis device 10 in accordance with the first aspect of the present disclosure. The fourth portion 400 is configured to provide support to the palm of the patient, so that the hand of the patient remains steady while performing the flexion or expansion movement. The fourth portion 400 comprises a plurality of provisions 52 for attachment of the fourth portion to the third portion. The plurality of through holes are configured to be attached to the protrusion of the extended portion of the third portion 300 for providing secure connection between the third portion 300 and the fourth portion 400. The fourth portion 400 further comprises a plurality of guide holes 51 corresponding to the hollow pipe portions and the back hole of the third portion.

The device 10 further comprises a guide structure for proper movement of the body portion. The guide structure comprises a plurality of wires 20 passing through the support structure 1 such that the plurality of wires 20 provide movement such as expansion and flexion of the fingers of the hand. In an embodiment, the plurality of wires 20 are attached with the at least one actuator 2 and are configured to move, i.e., wind and unwind, in accordance with the force, in the clockwise direction and anti-clockwise direction, generated by the at least one actuator 2. The plurality of wires 20 pass through the first set and the second set of holes 23, 24 of the first portion 100, second set of holes 33, 34, 35 of the second portion 200, and the plurality of guide holes 51 of the fourth portion 400, to provide accurate movement fingers of the hand of the patient. The winding and unwinding of the plurality of wires 20 result in different states of the fingers of the hand, such as the state of flexion or the state of expansion. Further, if the EMG detected by the plurality of sensors 4, 5 is for relaxed state of the hand, the second set of motion signals generated by the control unit 3 corresponds to such state and the force generated by the actuator 2 corresponds to the relax state of the guide structure, and thus, of the hand. In an embodiment, the plurality of wires 20 are made of plastic. In other embodiment, the plurality of wires 20 are made of steel. Accordingly, the device of the present disclosure is low-cost device.

One use of the device 10 in accordance with the present disclosure is to help people with ulnar, medial, and radial nerve disabilities to perform proper hand movements. The design of the orthosis allows people to perform activities of daily living to improve their quality of life. Accordingly, the device represents a low-density myoelectric control system to control motor movement of body parts using myo-electric signals of human body.

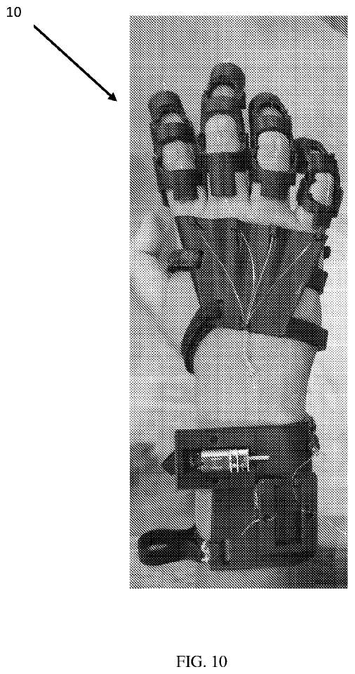

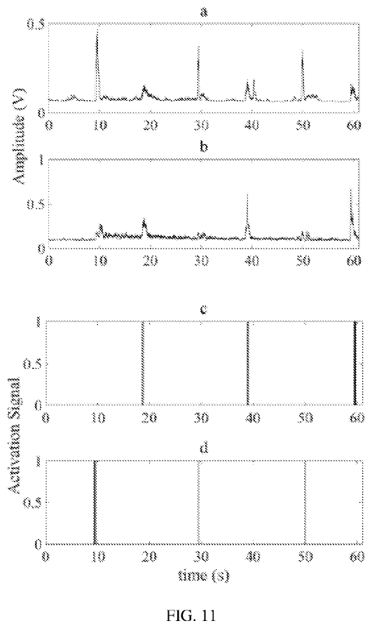

FIG. 10 illustrates the total structure of the orthosis comprising a first portion 100, a second portion 200, a third portion 300, a fourth portion 400 and a plurality of cables 20 integrated in the human hand complementing the movements thereof, in the part of the forearm actuator 2 is located.

FIG. 10 illustrates exemplary signals indicating the first set of motion signals and the corresponding second set of motion signals detected and generated by the orthosis device 10 in one of the experiments. The ON-OFF technique was used for generating the second set of motion signals. FIG. 10a and FIG. 10b illustrate the sensed first set of motion signals, especially the myoelectric signals of the flexor muscle are shown FIG. 10a, of the extensor muscle in FIG. 10b. FIG. 10c illustrates the anti-clockwise activation of the at least one actuator 2 to achieve finger flexion movement and FIG. 10d illustrates the clockwise activation of the at least one actuator 2 to achieve the movement finger extension movement with the orthosis device 10.

According to a second aspect of the orthosis device 10 of the present disclosure discloses a method for providing motor movement to a body part. The method is being performed by the orthosis device 10 as explained and discussed hereinabove. Accordingly, the method is performed by different components of the device 10 of the present disclosure. The method comprises the step of receiving, by a control unit 3, a first set of motion signals from a plurality of sensors 4, 5 placed on a preselected body part; processing, by the control unit 3, the received first set of motion signals; generating, by the control unit 3, a second set of motion signals based on the processed first set of motion signals; receiving, by at least one actuator 2, the second set of motion signals from the control unit 3; and displacing, by the at least one actuator 2, a support structure 1 to provide movement to the body part, the support structure 1 being configured to provide support to the body part.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

1. An orthosis device for providing motor movement to a body part, the device comprising:
a plurality of sensors configured to be placed on a preselected body part and further configured to detect a first set of motion signals from the preselected body part;
a control unit in communication with the plurality of sensors, the control unit configured to receive the first set of motion signals from the plurality of sensors and further configured to generate a second set of motion signals based on the received first set of motion signals;
at least one actuator in communication with the control unit, the at least one actuator configured to receive the second set of motion signals to be activated; and
a support structure in communication with the at least one actuator, the support structure configured to provide support to the body part and further configured to displace the body part in accordance with the at least one actuator, thereby providing motor movement to the body part.

2. The device of claim 1, wherein the support structure comprises a guide structure having a plurality of wires to facilitate movement of the body part.

3. The device of claim 1, wherein the at least one actuator is configured to provide movement to the support structure in a plurality of states.

4. The device of claim 1, wherein the plurality of sensors are non-invasive sensors.

5. The device of claim 2, wherein the at least one actuator is configured to generate a force to displace the guide structure of the support structure, thereby providing movement to the body part.

6. The device of claim 1, wherein the control unit is configured to:
receive the first set of motion signals;
classify the first set of motion signal into a class of a plurality of classes; and
generate a corresponding second set of motion signals based on the class.

7. The device of claim 6, wherein the plurality of classes corresponds to a plurality of states of movement of the support structure, that comprises a state of flexion, a state of expansion, and a relax state.

8. The device of claim 1, wherein the at least one actuator is configured to generate a force in the clockwise direction and the anticlockwise direction, wherein least one actuator is a servo motor.

9. The device of claim 2, wherein the support structure further comprises a first portion, a second portion, a third portion, and a fourth portion.

10. The device of claim 9, wherein the first portion comprises a top portion to provide support to a distal end of the body part and to prevent hypertension while movement, a first protrusion and a second protrusion configured to be connected with the second portion and further configured to form a joint, a first set of holes to provide expansion to the body part, and a second set of holes to provide flexion to the body part.

11. The device of claim 10, wherein the second portion comprises a plurality of articulation holes configured to connect the first protrusion and the second protrusion of the first portion, a first set of protrusions to connect to the third portion, and a second set of holes configured to facilitate the guide structure to pass therewithin.

12. The device of claim 11, wherein the third portion comprises a top edge having a plurality of holes configured to be connected to the first set of holes of the second portion, and a bottom edge with an extended portion to connect to the fourth portion, which is configured to provide support to the guide structure.

13. A method for providing motor movement to a body part, the method comprising:
receiving, by a control unit, a first set of motion signals from a plurality of sensors placed on a preselected body part;
processing, by the control unit, the received first set of motion signals;
generating, by the control unit, a second set of motion signals based on the processed first set of motion signals;
receiving, by at least one actuator, the second set of motion signals from the control unit; and
displacing, by the at least one actuator, a support structure to provide movement to the body part, the support structure being configured to provide support to the body part.

14. The method of claim 13, wherein the step of processing comprises the step of classifying the first set of motion signals into a class of a plurality of classes.

15. The method of claim 14, wherein the plurality of classes corresponds to a plurality of states of movement of the support structure., wherein comprises a state of flexion class, a state of expansion class, and a relax state class.
